(19) United States
(12) Patent Application Publication
(10) Pub. No.: US 2010/0137975 A1
(43) Pub. Date: Jun. 3, 2010

(54) ACTIVE-SUBSTANCE-COATED MEDICAL PRODUCT, METHOD FOR ITS PRODUCTION AND ITS USES

(75) Inventor: Eric Wittchow, Nuernberg (DE)

Correspondence Address: BIOTECH BEACH LAW GROUP , PC, 5677 OBERLIN DRIVE, SUITE 204, SAN DIEGO, CA, 92121, US

(73) Assignee: BIOTRONIK VI PATENT AG, Baar (CH)

(21) Appl. No.: 12/630,177

(22) Filed: Dec. 3, 2009

(30) Foreign Application Priority Data
Dec. 3, 2008 (DE) 10 2008 044 316.6

(51) Int. Cl. A61F 2/06; A61F 2/00; A61K 9/50; A61F 2/82; A61B 17/22

(52) U.S. Cl. 623/1.38; 424/423; 424/501; 623/1.42; 606/159

(57) ABSTRACT

The present invention relates to a medical product that can be implanted or introduced into a vascular system of a human or animal organism, having an active substance coating according to the invention on the surface of the medical product, to a method for the production of a medical product that can be implanted or introduced into a vascular system of a human or animal organism, having an active substance coating according to the invention, to an active substance coating according to the invention for a medical product that can be implanted or introduced into a vascular system of a human or animal organism, to uses of the active substance coating according to the invention for the production of a medical product that can be implanted or introduced into a vascular system of a human or animal organism, and to a method for treatment of a stenosis, etc., in the vascular system of a human or animal organism.

### CROSS REFERENCE TO RELATED APPLICATIONS

This invention claims benefit of priority to Germany patent application serial number DE 10 2008 044 316.6, filed on Dec. 3, 2008; the contents of which is herein incorporated by reference in its entirety.

### FIELD OF THE INVENTION

The present invention relates to a medical product that can be implanted or introduced into a vascular system of a human or animal organism, having an active substance coating according to the invention on the surface of the medical product, to a method for the production of a medical product that can be implanted or introduced into a vascular system of a human or animal organism, having an active substance coating according to the invention, to an active substance coating according to the invention for a medical product that can be implanted or introduced into a vascular system of a human or animal organism, to uses of the active substance coating according to the invention for the production of a medical product that can be implanted or introduced into a vascular system of a human or animal organism, and to a method for treatment of a stenosis, etc., in the vascular system of a human or animal organism.

### BACKGROUND OF THE INVENTION

In general, medical products that can be implanted or introduced into the vascular system, such as stents or angioplasty balloons, are used for the treatment of stenoses, particularly by means of the angioplasty balloon method.

Stents, in general, are endovascular (peripheral or coronary) prostheses or implants that are known, for example, for the treatment of stenoses, but also for the treatment of aneurysms.

Stents fundamentally have a support structure that is suitable for supporting the wall of a blood vessel in suitable manner, in order to thereby open the blood vessel or to bridge an aneurysm. For this purpose, stents are introduced into the blood vessel in a compressed state, then expanded at the location to be treated, and pressed against the blood vessel wall. This expansion can take place, for example, using an angioplasty balloon, affixed to an insertable catheter. Alternatively, self-expanding stents are also known. These are composed of a super-elastic metal, such as nitinol, for example.

Stents are currently divided up into two basic types, permanent stents and degradable stents. Permanent stents are configured in such a manner that they can remain in the blood vessel for an indefinite period of time. Degradable stents, on the other hand, are decomposed in a blood vessel, over a pre-determined period of time.

In order for expansion of the blood vessels by means of stent implantation, so-called stenting, to be successful, particularly also within the scope of an angioplasty balloon method, a stent or an angioplasty balloon should be selected in such a manner that vascular occlusion does not occur again in the region of the inserted stent.

Usually, for this purpose, active-substance-coated stents and angioplasty balloons are used, where the active substances either accelerate the healing process or counteract irritations that might occur due to the implanted stent or the inserted angioplasty balloon.

It is therefore desirable that these active substances can maintain their active properties over as long a period of time as possible, so that healing of the blood vessel wall and reduction of the side effects is supported over as long a period of time as possible.

It is therefore the task of the present invention to make available an improved active substance coating for a medical product to be implanted or introduced into a blood vessel, where the coating is supposed to be easy to produce, is supposed to provoke (essentially) no undesirable physiological reactions, is supposed to demonstrate sufficient mechanical properties, i.e. so that a stent can be coated before being crimped, and has an (essentially) functional coating after crimping, and/or the coating is supposed to demonstrate sufficient scratch resistance so that it is not damaged, particularly during and after introduction into the human or animal organism by means of a guide catheter, and/or the duration of the active substance effect is present in elevated form, as compared with conventional active substance coatings.

### SUMMARY OF THE INVENTION

The present task is completely or partly accomplished by means of the objects of the independent claims, according to the invention.

Accordingly, a first embodiment according to the invention relates to a medical product that can be implanted or introduced into a vascular system of a human or animal organism, having an active substance coating on the surface of the medical product, characterized in that the active substance coating includes one or more separate active substance particles, including one or more active substances and one or more polymers, having a particle diameter less than or equal to 30 μm, the active substance particle(s) can be detached from the coating of the medical product under physiological conditions in the vascular system, and the detached active substance particle(s) essentially does/do not release the active substance(s) at a pH in the range of 7.3-7.5.

A second embodiment according to the invention relates to a method for the production of a medical product that can be implanted or introduced into a vascular system of a human or animal organism, having an active substance coating, characterized in that: (a) one or more separate active substance particles is/are made available, which include one or more active substances and one or more polymers, and have a particle diameter less than or equal to 30 μm, (b) a base body of a medical product is made available, and (c) the base body of step (b) is coated with the active substance particles of step (a), in such a manner that the active substance particles can be detached under physiological conditions in the vascular system, in the coated state, but the active substance(s) is/are essentially not released from the detached active substance particles at a pH in the range of 7.3 to 7.5.

A third embodiment according to the invention relates to an active substance coating for a medical product that can be implanted or introduced into a vascular system of a human or animal organism, characterized in that the active substance coating includes one or more separate active substance particles including one or more active substances and one or more polymers, having a particle diameter less than or equal to 30 μm, the active substance particle(s) can be detached under physiological conditions in the vascular system, from the coating of the medical product, and the detached active substance particle(s) essentially does/do not release the active substance(s) in a pH range of 7.3-7.5.

A fourth embodiment according to the invention relates to use of the active substance coating according to the invention for the production of a medical product that can be implanted or introduced into a vascular system of a human or animal organism.

A fifth embodiment according to the invention relates to a method for treatment of a stenosis, a calcified or soft plaque, or an aneurysm in the vascular system of a human or animal organism, characterized in that an active-substance-coated medical product according to the invention is made available, and implanted or introduced into the vascular system of the human or animal organism.

Preferred embodiments of the objects according to the invention are presented in the dependent claims and in the following detailed description, and can be combined with one another, to the extent that this makes sense.

### DETAILED DESCRIPTION OF THE INVENTION

The present invention is based on the surprising recognition that one or more separate active substance particle(s) of the active substance coating according to the invention, having a particle diameter less than or equal to 30 μm, are detached from the coating of a medical product according to the invention, after implantation or introduction into a vascular system of a human or animal organism, preferably at the location of interest, i.e. in the region of a stenosis, a calcified or soft plaque, or an aneurysm in the vascular system of a human or animal organism, where the detached active substance particles are recognized by the organism as foreign bodies, and phagocytized by the body's own macrophages.

In this connection, the macrophages first form pseudopods (plasma evulsions) and preferably enclose the foreign body completely with them. In this process, a vacuole, the so-called phagosome, is formed. It is characterized by a diameter of approximately 30 μm, so that the detached active substance particle to be used according to the invention, having a particle diameter of less than or equal to 30 μm, can preferably be completely taken up. Usually, a pH of about 6.5 prevails in the phagosome. The vacuole then merges with the macrophage lysosomes, and a phago-lysosome is formed, which contains not only reactive chemical species (RCS), for example NO, H₂, O₂, O₂⁻, but also lytic enzymes, and in which a pH of about 5 prevails.

The active substance particles to be used according to the invention, including one or more active substance and one or more polymers, are adjusted in such a manner that they (essentially) do not release the active substance(s) at a physiological pH in the range of 7.3 to 7.5. According to the invention, “do not release” or “essentially do not release” means that preferably, 60 wt.-% or more of the active substance, with reference to the weight of the active substance in the active substance particle, are not released at a pH in the range of 7.3-7.5 within the first 24 hours. In a preferred embodiment, the active substance(s) is/are released from the active substance particle at a pH that prevails in the phagosome or the phago-lysosome, i.e. preferably at a pH in the range of 6.5 or less, furthermore preferably in the range of 5-3.

An adjustment of the release speed of the active substance(s) from the active substance particle to be used according to the invention can furthermore be carried out by way of the solubility of the active substance(s) and, if necessary, by way of the size of the active substance particle to be used according to the invention. Since, according to the invention, the saturation point and therefore the rate of release of the active substance lies higher in the case of the lysosomal pH than in the case of the cytoplasmatic pH, the size of the active substance particles to be used according to the invention can also be decisive for the release kinetics, because for quick release, the macrophage should preferably enclose the active substance particle completely. In other words, the smaller the active substance particle, usually the faster the decomposition that takes place. Preliminary in vitro experiments have shown that the active substance particles detached from the coating are taken up by the macrophages within a few hours to one day. According to the invention, the active substance particles subsequently release the active substance(s) in the phagosome or phago-lysosome. For this reason, the average particle diameter preferably lies in the range of 0.01 to 10 μm, furthermore preferably 0.1 to 5 μm, and very particularly preferably 80 to 500 nm. When the local concentration of the active substance(s) in the phagosome or phago-lysosome reaches a critical value, the phagosome or phago-lysosome is destroyed, and releases the active substance(s), to preferably act at the location of interest.

Thus, the macrophages can serve, along with the medical product itself, as a further storage unit for the active substance particles. Subsequently, the active substance(s), which can then act directly at the location of interest, are released in the storage unit. This is of particular interest in the case of those active substances that otherwise demonstrate too short a dwell time in the tissue, because of their hydrophilia. Therefore, an extension and/or elevation of the local concentration of the active substances can occur, according to the invention, as compared with conventional medical products coated with active substances, particularly at the location of interest, because after completion of the release of the active substance particles from the coating of the medical product, first the active substance particles are stored in the macrophages, and only there do they release the active substance(s). In the case of conventional medical products coated with active substances, in contrast to the present invention, the active substances are usually released directly from the active substance coating, and are regularly transported away with the blood stream, so that the duration of effect and the active concentration can be lower as compared with the present invention.

An active substance particle to be used according to the invention is preferably structured in such a way that the polymer(s) is/are selected from among pH-sensitive degradable polymers. In another preferred embodiment, the active substance(s) is/are sheathed (encapsulated) by a pH-sensitive degradable polymer mixture, or are present incorporated into its matrix. In a particularly preferred embodiment, they are present in encapsulated form. Such preferred active substance particle embodiments, by means of the selection of one or more correspondingly suitable pH-sensitive degradable polymers, permit the active substance(s) to be essentially not released (or released slowly) at a pH in the range of 7.3-7.5, but to be released (more quickly) at a pH in the range of 6.5 or less, preferably 5 to 3. Suitable polymer materials degrade faster, accordingly, at a pH in the range of 6.5 or less, preferably 5 to 3, as compared with a pH in the range of 7.3-7.5. Accordingly, they are preferably selected from among the group including poly-β-amino esters), poly-(ethylene oxide) modified poly-β-amino esters), polylactides, poly-(lactide-co-glycolides), modified cyclodextrins, modified carbohydrates and/or other polyesters.

Poly-β-amino ester) active substance particles that are suitable according to the invention, as well as their production methods, are described, for example, by David M. Lynn et al. (“pH-Responsive Polymer Microspheres: Rapid Release of Encapsulated Material within the Range of Intracellular pH,” Angew. Chem. Int. Ed.; 2001; 40; No. 9; p. 1707-1710).

Poly-(ethylene oxide)-modified poly-β-amino ester) active substance particles that are suitable according to the invention, as well as their production methods, are described in, among others, Shenoy et al. (“Poly-(Ethylene Oxide)-modified Poly-(β-Amino-Ester) Nanoparticles as a pH-Sensitive System for Tumor-Targeted Delivery of Hydrophobic Drugs: Part 1. In vitro Evaluations”, Mol. Pharm. 2005: 2(5): 357-366, see also (“Poly-(Ethylene Oxide)-modified Poly-(β-Amino-Ester) Nanoparticles as a pH-Sensitive System for Tumor-Targeted Delivery of Hydrophobic Drugs Part 2. In vivo Distribution and Tumor Localization Studies,” Pharm Res.; 2005 December; 22(12): 2107-2114) as well as by A. Potineni et al. (“Poly-(ethylene oxide)-modified poly-β-amino-ester) nanoparticles as a pH-sensitive biodegradable system for paclitaxel delivery,” J. Controlled Release; 86 (2003); 223-234).

Polylactide and poly-(lactide-co-glycolide) active substance particles that are suitable according to the invention, as well as their production methods, are described, for example, by H. Fessi et al. (“Nanocapsule formation by interfacial polymer deposition following solvent displacement,” Int. J. Pharmaceutics; 55 (1989); R1-R4), M. Koneracká et al. (“Encapsulation of anticancer drug and magnetic particles in biodegradable polymer nanospheres,” J. Phys.: Condens. Matter; 20 (2008); 204151 (6 pp)), D. Klose et al. (“PLGA-based drug delivery systems: Importance of the type of drug and device geometry”; Int. J. Pharmaceutics; 354 (2008); 95-103), and Kang et al. (“Preparation of PLLA/PLGA microparticles using solution enhanced dispersion by supercritical fluids (SEDS),” J. Colloid and Interface Science; 322 (2008); 87-94).

Cyclodextrin active substance particles that are suitable according to the invention, as well as their production methods, are described, for example, by Erem Memisoglu et al. (“Amphiphilic β-Cyclodextrins Modified on the Primary Face: Synthesis, Characterization, and Evaluation of Their Potential as Novel Excipients in the Preparation of Nanocapsules”; J. Pharm. Sciences; Vol. 91; No. 5; May 2002) as well as by Erem Bilensoy et al. (“Safety and efficacy of amphiphilic β-cyclodextrin nanoparticles for Paclitaxel delivery”; Int. J. Pharmaceutics; 347 (2008); 163-170).

The active substance particles to be used according to the invention, listed as examples above, are particularly suitable for the use of active substances having hydrophobic surface properties.

The active substance particles to be used according to the invention are applied to the surface of the medical product by means of usual methods. In this connection, the active substance particles can preferably be present (a) incorporated into a degradable matrix or (b) as an active substance coating on the surface of the medical product by means of an adhesive layer, in order to be able to be detached from the active substance coating of the medical product under physiological conditions, preferably at a pH of 7.0 to 7.5.

If the active substance particle(s) is/are present (a) incorporated into a degradable matrix, then they are released step by step, on the basis of erosion/degradation of the matrix material. Consequently, the materials of the degradable matrix are selected in such a way that they degrade or erode more rapidly than the polymer materials of the active substance particle, under physiological conditions, preferably at a pH in the range of 7.0 to 7.5. According to this preferred embodiment according to the invention, the degradable matrix forms a first storage medium for the active substance particles, and the macrophages form a second storage medium. By means of this preferred embodiment, the duration of effect of the active substance(s) can be extended. In another preferred embodiment of this, the degradable matrix is selected from among one or more materials from the group including polydioxanone; polycaprolactone, polyglycolides; polylactides, preferably poly-(L-lactide), poly-(D-lactide), poly-(D,L-lactide), as well as blends, copolymers and tripolymers of them, preferably poly-(L-lactide-co-glycolide), poly-(D-L-lactide-co-glycolide), poly-(L-lactide-co-L-lactide), poly-(L-lactide-co-trimethylene carbonate); polysaccharides, preferably chitosan, levan, hyaluronic acid, heparin, dextran, and celluloses; polyhydroxyvalerate; ethyl vinyl acetate; polyethylene oxides; polyphosphoryl choline; fibrin; albumin; and/or polyhydroxybutyric acids, preferably atactic, isotactic and/or syndiotactic polyhydroxybutyric acid as well as their blends.

If the active substance particle(s) is/are (b) coated onto the surface of the medical product, so as to be detachable under physiological conditions, then preferably, the bond between active substance particle and adhesion material is degraded more quickly under physiological conditions, preferably at a pH in the range of 7.0 to 7.5 (usually, the adhesive material degrades), than the polymer of the active substance particles degrades and releases the active substance(s).

Usually, however, the bond of the adhesive material to the active substance particles is so strong that upon implantation or introduction of the medical product, the active substance coating on the medical product according to the invention is not or not significantly functionally impaired. Accordingly, all physiologically suitable materials that degrade more quickly than the polymer in the active substance particle, under physiological conditions, are preferred as adhesive materials. Particularly preferred adhesive materials are selected from among the group including carbohydrates, preferably sugar and/or polysaccharides, preferably starches and/or hyaluronic acid; (soy) lecithin; (oligo)peptides and/or quickly degradable polyesters.

An active substance in the sense of this invention is a substance or a compound that provokes a biological reaction in the human or animal body. In this sense, the term active substance can also be used synonymously with medicinal substance and/or pharmaceutical. In the sense of the present invention, the medical product according to the invention, preferably a stent or an angioplasty balloon, is coated with one or more active substances in a concentration that is sufficient to provoke the desired physiological reactions.

Active substances to be used according to the invention are preferably selected from among the group including antiphlogistics, preferably dexamethasone, methyl prednisolone, and diclofenac; cytostatics, preferably paclitaxel, colchicine, actinomycin D, and methotrexate; immune suppressives, preferably limus compounds, particularly preferably sirolimus (fapamycin), zotarolimus (ABT-578), tacrolimus (FK-506), everolimus, biolimus, particularly biolimus A9 and pimecrolimus, cyclosporin A, and mycophenolic acid; thrombocyte aggregation inhibitors, preferably abciximab and iloprost; statins, preferably simvastatin, mevastatin, atorvastatin, lovastatin, pitavastatin, and fluvastatin; estrogens, preferably 17b-estradiol, daidzein and genistein; lipid regulators, preferably fibrates; immune suppressives; vasodilators, preferably satanes [sic—apparent typo in the German, intended meaning not evident]; calcium channel blockers; calcineurin inhibitors, preferably tacrolimus; anti-inflammatories, preferably imidazoles; anti-allergies oligonucleotides, preferably decoy-oligodesoxynucleotide (dODN); endothelium-forming agents, preferably fibrin; steroids; proteins/peptides; proliferation inhibitors; analgesics and anti-rheumatics; endothelin receptor antagonists, preferably bosentan; rho-kinase inhibitors, preferably fasudil; RCD-peptides (including the sequence Arg-Gly-Asp); and/or organic gold compounds.

In the sense of the present invention, medical products can be (i) implanted or (ii) introduced into a vascular system of a human or animal body.

Usual (i) implantable medical products (implants or implant base bodies) can represent all medical, plastic and/or functional implants or implant base bodies that are implanted into a blood vessel of a human or animal organism and release (elute) active substances. An active-substance-eluting stent is particularly preferred as a medical product according to the invention.

Usually, the original mechanical functions of the implantable medical products according to the invention are supposed to be maintained, i.e. in the case of a coronary stent, for example, its dilatability, its low recoil, its stability over a desired time period (in the case of a degradable stent), as well as its flexibility.

In the following, implant materials, preferably stent base body materials, as well as preferred embodiments of them, usually to be used according to the invention, will be described:

### Degradable Implant Base Body, Particularly Degradable Stent Base Body:

In the sense of the present invention. “degradable implant (base body),” particularly “degradable stent (base body),” means that the base body degrades in a physiological environment, particularly in the vascular system of a human or animal organism, i.e. is decomposed in such a manner that the stent loses its integrity. Preferably, degradable implant base bodies are only decomposed once the function of the implant is no longer physiologically useful or necessary. In the case of degradable stents, this means that the stent is preferably degraded or loses its integrity once the traumatized tissue of the blood vessel has healed and the stent therefore no longer has to remain in the blood vessel lumen.

### Metallic Base Body:

In an embodiment according to the invention, the degradable material includes a metallic material that represents a biocorrodible alloy, where the main component of the alloy is selected from among the group of magnesium, iron, zinc and/or tungsten; in particular, a magnesium alloy or an iron alloy is preferred for a degradable metallic material, particularly preferably a magnesium alloy.

The alloy, particularly including magnesium, iron, zinc and/or tungsten, should be selected, in terms of its composition, in such a manner that it is biocorrodible. Alloys that undergo decomposition in a physiological environment, which leads, in the final analysis, to the part of the stent formed from the material losing its mechanical integrity, are referred to as biocorrodible in the sense of the present invention. In the present case, an alloy is understood to be a metallic structure whose main component is magnesium, iron, zinc and/or tungsten. The main component is the alloy component whose weight proportion in the alloy is the greatest. A proportion of the main component preferably amounts to more than 50 wt.-%, furthermore preferably more than 70 wt.-%. A magnesium alloy or an iron alloy is preferred. A magnesium alloy is particularly preferred.

If the material is a magnesium alloy, this preferably contains yttrium and other rare earth metals, since such an alloy is characterized by its physical chemistry properties and its great biocompatibility, particularly also of its decomposition products.

Magnesium alloys of the WE series, particularly WE43, as well as magnesium alloys having the composition rare earth metals 5.5-9.9 wt.-%, of this yttrium 0.0-5.5 wt.-% and the remainder <1 wt.-%, where the remainder can contain zirconium and/or silicon and where magnesium takes up the remaining proportion of the alloy, to come to 100 wt.-%, are preferably used. These magnesium alloys have already confirmed their particular suitability in experiments and in preliminary clinical trials, i.e. they demonstrated great biocompatibility, advantageous processing properties, good mechanical characteristics, and a corrosion behavior that was adequate for the purposes of use. In the present case, the general term “rare earth metals” is understood to include scandium (21), yttrium (39), lanthanum (57), and the 14 elements that follow lanthanum (57), namely cerium (58), neodymium (60), promethium (61), samarium (62), europium (63), gadolinium (64), terbium (65), dysprosium (66), holmium (67), erbium (68), thulium (69), ytterbium (70), and lutetium (71).

### Polymer Base Body:

Implant (base bodies), particularly stent (base bodies), according to another alternative embodiment according to the invention, can include degradable polymer, preferably selected from among the group that includes: polydioxanone; polycaprolactone, polyhydroxyvalerianic acid; polyhydroxybutyric acid; polyglycolides; polylactides, preferably poly-(L-lactide), poly-(D-lactide), poly-(D,L-lactide), as well as blends, copolymers and tripolymers of them, preferably poly-(L-lactide-co-glycolide), poly-(D-L-lactide-co-glycolide), poly-(L-lactide-co-L-lactide), poly-(L-lactide-co-trimethylene carbonate); polysaccharides, preferably chitosan, levan, hyaluronic acid, heparin, dextran and celluloses; phosphazenes; polyphosphoesters; polyphosphonates and polyphosphites.

### Permanent Implant (Base Body), Preferably Permanent Stent (Base Body):

In contrast to degradable base bodies, a permanent implant (base body), particularly a permanent stent (base body), is essentially not decomposed in a physiological environment in the human or animal organism, i.e. it maintains its integrity.

In another embodiment according to the invention, the base body of a permanent implant, particularly of a permanent stent, preferably includes a shape memory material having one or more materials selected from among the group of nickel-titanium alloys and copper-zinc-aluminum alloys, preferably of nitinol.

In a particularly preferred embodiment according to the invention, the base body of a permanent implant, particularly of a permanent stent, includes stainless steel, preferably of a Cr—Ni—Fe steel, here preferably the alloy 3161, or a Co—Cr steel.

In another preferred embodiment, the base body of the implant, preferably the stent, can additionally include plastic, preferably polyurethane, and/or ceramic and/or other polymer coatings.

If, in the sense of the present invention, endovascular implantable stents are used as implantable medical products, then all the usual stent geometries can be used. Stent geometries that are particularly described in U.S. Pat. No. 6,896,695, US 2006/241742, U.S. Pat. No. 5,968,083 (Tenax), EP 1 430 854 (Helix-Design), U.S. Pat. No. 6,197,047, and EP 0 884 985 are particularly preferred.

A peripheral or coronary stent according to the invention is preferably coated, on the mural side, with the active substance coating according to the invention, i.e. in the case of a usual cylindrical stent geometry, the surface that stands in contact with the tissue and not with the vascular lumen of the blood vessel after implantation. Such a preferred coating, according to the invention, can contribute to the fact that fewer side effects are provoked. In the case of a degradable base body, this preferred coating furthermore allows the decomposition of the luminal surface of the stents, i.e. of the surface that stands in contact with the lumen of the blood vessel in the case of the usual cylindrical stent.

In another embodiment according to the invention, (ii) medical products that can be introduced represent active-substance-eluting angioplasty balloons. For this purpose, usual, mass-produced balloon catheters are used, which are preferably “compliant,” i.e. their diameter changes, over a relatively broad range, with increasing pressure. These usual balloon catheters are coated, according to the invention, in order to obtain a medical product that can be introduced, according to the invention.

In another preferred embodiment, a medical product according to the invention can additionally have one or more coatings (free of active substance) on the active substance coating to be used according to the invention, as a so-called “top coat,” particularly in order to reduce the risk of abrasion of (part of) the active substance coating during implantation or introduction of the medical product into the vascular system.

For the top coat, according to the invention, usually one or more polymers can be used, which are preferably selected from among the group including (a) non-degradable polymers: polyethylenes; polyvinyl chlorides, polyvinyl fluorides, polyvinyl alcohols; polyacrylates, preferably polyethyl acrylates and polymethacrylates; polymethyl methacrylate, polymethyl-co-ethyl acrylates and ethylene/ethyl acrylates; polytetrafluoroethylene, preferably ethylene/chlorotrifluoroethylene copolymers, ethylene/tetrafluoroethylene copolymers; polyamides, preferably polyamide imide, PA-11, PA-12, PA-46, PA-66; polyether imides; polyether sulfones; poly(iso)butylenes; polyurethanes; polybutylene terephthalates; silicones, polyphosphatanes; polymer foams, preferably polymer foams composed of carbonates, styrenes; copolymers and/or blends of the polymer classes, polymers of the class of the thermoplastics, and/or (b) degradable polymers: polydioxanones; polycaprolactones, polyhydroxy valeric acid (derivatives); polyglycolides; polylactides, preferably poly-(L-lactide), poly-(D-lactide), poly-(D,L-lactide), as well as blends, copolymers and tripolymers of them, preferably poly-(L-lactide-co-glycolide), poly-(D-L-lactide-co-glycolide), poly-(L-lactide-co-L-lactide), poly-(L-lactide-co-trimethylene carbonate); ethyl vinyl acetate; polyethylene oxide, polyphosphoryl choline; fibrin; albumin; polyhydroxybutyric acid, preferably atactic, isotactic and/or syndiotactic polyhydroxybutyric acid as well as their blends.

Particularly preferred polymers for the “top coat” of the present invention are the degradable polymers described above, because no component that is foreign to the body remains in the organism, as a result of the complete decomposition of the polymer(s).

According to a second embodiment according to the invention, a method for the production of a medical product that can be implanted or introduced into a blood vessel of a human or animal organism, having an active substance coating, is claimed. Preferably, a method for the production of a medical product according to the invention is claimed. The preferred embodiments of the medical product according to the invention or the active substance coating according to the invention also apply to the method for the production of a medical product according to the invention, and can be combined in any manner that makes sense to a person skilled in the art.

With regard to the configuration according to the invention regarding the production method, according to method step a), active substance particles to be used according to the invention are made available. Corresponding methods for the production of active substance particles to be used according to the invention are known to a person skilled in the art, and have furthermore already been described above, as examples. In this connection, Kang et al. (ibid.) and Potineni et al. (ibid.) describe the production of nanoparticles including an active substance sheathed in a polymer, from supercritical fluids. The production of active substance nanoparticles by means of precipitation in water is described, for example, in Bilensoy et al.

According to step b) of the production method according to the invention, a base body of the medical product is made available. Base bodies to be used according to the invention were already described with regard to the medical products according to the invention, particularly implants and implant base bodies, preferably stents and stent base bodies, as well as with regard to the medical products that can be introduced, preferably angioplasty balloons. A stent base body is particularly preferred as an implant base body, and, in this connection, in particular, a degradable stent base body is used, as was already described above.

According to step c) of the production method according to the invention, the base body from step b) is coated with the active substance particles from step a), in such a manner that the active substance particle(s) can be detached from the coating in the coated state, under physiological conditions, preferably at a pH in the range of 7.0-7.5 in the vascular system.

Such an active substance coating according to the invention can be produced using usual methods, where it is advantageous to apply a dry mixture of active substance particles, a mixture of active substance particles/solvent/binder (solvent is suitable for (partially) dissolving the binder, but not the materials of the active substance particles), or a mixture of active substance particles/polymer, for example by means of a dipping method (dip coating), by means of spray coating using a single-substance or multi-substance nozzle, by means of rotation atomization, and by means of sputtering, onto the surface of the medical product, which has been pretreated, if necessary.

If, according to the invention, a dry mixture of active substance particles is used, then before the medical product is coated with the active substance particles to be used according to the invention, the corresponding surface of the medical product is pretreated with an adhesion material, in such a way that in a subsequent step, the active substance particles to be used according to the invention adhere to the surface of the medical product in such a manner that the active substance particles are detached from the surface of the medical product under physiological conditions, preferably at a pH in the range of 7.0-7.5. In the case of a spraying method, the active substance particles to be used according to the invention usually have a particle diameter that is smaller than the nozzle diameter. Similar coating methods can also usually be used for coating with the “top coat.”

For the case that preferably, only the mural surface of a stent according to the invention is supposed to be coated with an active substance coating according to the invention, this can preferably be done in that during the aforementioned method steps, the stent is set onto a cylinder, a cannula, or a mandrel, for example, so that only the mural surface of the stent is coated with the active substance coating according to the invention. Alternatively, the mural surface could be coated with the active substance coating according to the invention by means of roller application or by means of selective application using brushing, filling of cavities, etc. Similar methods can also preferably be used for the “top coat coating.”

If necessary, a usual drying step, or other usual physical or chemical subsequent processing steps, for example vacuum treatments or plasma treatments, can follow one or more coating steps, before the medical product according to the invention, preferably a stent or angioplasty balloon, is treated further.

The fourth embodiment according to the invention, namely the use of the active substance coating according to the invention for the production of a medical product that can be implanted or introduced into the vascular system of a human or animal organism, is preferably directed at the use of the active substance coating according to the invention for the production of a medical product according to the invention. The preferred embodiments of the medical product according to the invention and the active substance coating according to the invention, respectively, accordingly apply also for the fourth embodiment, and can be combined with one another, to the extent that this appears practical to a person skilled in the art.

In a preferred embodiment, the active substance coating according to the invention or the medical product according to the invention is used to extend the duration of effect of one or more active substances in the vascular system of a human or animal organism. Here again, the preferred embodiments of the medical product according to the invention and of the active substance coating according to the invention, respectively, can be applied to its use, and can be combined, to the extent that this is practical.

According to the fifth embodiment according to the invention, a treatment method is claimed, which is characterized in that a medical product coated with active substance according to the invention is made available and implanted or introduced into the vascular system of the human or animal organism. Here again, the preferred embodiments of the medical product according to the invention and of the coating according to the invention, respectively, can be used, and can be combined, to the extent that this appears practical to a person skilled in the art.

The preferred embodiments of the medical product that can be used according to the invention, preferably of the stent or angioplasty balloon according to the invention, as well as of the active substance coating according to the invention, can be combined with one another in all the variants that appear practical to a person skilled in the art, but also with the other preferred embodiments disclosed herein.

### EXAMPLES

In the following, the present invention will be described using exemplary embodiments, but these do not limit the scope of protection of the objects according to the invention.

### A) Production of Active Substance Particles to be Used According to the Invention

### Production Example A1

### Production of Paclitaxel-Charged Cyclodextrin Nanoparticles

A solution of 3 g 6-0-CAPRO-β-CD [heptakis-(6-deoxy-6-hexane amido) cyclomaltoheptanose; MW: 1820 g/mol] and 640 mg paclitaxel (MW: 854 g/mol) in 1000 ml acetone, in a 1:1 molar ratio, is produced at room temperature (RT), while stirring constantly. This organic solution is slowly placed into 2000 ml ultra-pure water, while stirring constantly, causing the clear solution to immediately have a cloudy appearance. The acetone is removed in a vacuum, with the nanoparticles, which have a size of approximately 150-500 nm, precipitating out completely. These are centrifuged off, rinsed with buffer solution, dried in a vacuum, and stored in a refrigerator at 8° C.

### Production Example A2

### Production of Sirolimus-Charged PLGA Nanoparticles

10 g PLGA [poly-(D,L-lactide-co-glycolide), glycolide proportion 85:15; MW: 50,000-75,000; from Sigma] and 200 mg sirolimus (MW: 914 g/mol; from Sigma) are dissolved in 1000 ml acetone, at RT, while stirring constantly, in order to produce the organic phase. The aqueous phase includes 10 g Pluronic®F68 (from BASF) as a surfactant, and 2000 ml ultra-pure water. The organic phase is added to the aqueous phase drop by drop, while stirring, causing a colloidal solution to form. The organic solvent and half of the water are removed under reduced pressure; the precipitated nanoparticles are centrifuged off, rinsed with a aqueous buffer solution, and freeze-dried.

### Production Example A3

### Production of Bosentan-Charged PEO-Modified PbAE Nanoparticles

A PEO-modified poly-β-amino ester) (PbAE) solution is produced according to the instructions of Lynn et al. (ibid.). The organic phase is produced by means of dissolving an equimolar amount of bosentan (an endothelin receptor antagonist; Tracleer®) and PEO-PbAE in absolute alcohol, in such a manner that a 1 mM solution is formed. The organic phase is added, drop by drop, at 15° C., into twice the volume of an aqueous solution of 0.1-1 wt.-% Pluronie®F108 (from BASF) in ultra-pure water, causing a colloidal solution to form. The organic solvent and half of the water are removed under reduced pressure, the precipitated nanoparticles are centrifuged off (10,000 rpm; 20 min), rinsed with an aqueous buffer solution, and freeze-dried.

### Production Example A4

### Production of Paclitaxel-Charged PLA Nanoparticles by Means of Dispersion in a Supercritical Fluid

5 g PLGA [poly-(D,L-lactide-co-glycolide), glycolide proportion 85:15; MW: 50,000-75,000; from Sigma] and 200 mg sirolimus (MW: 914 g/mol; from Sigma) are dissolved in 1000 ml dichloromethane, at RT, while stirring constantly. The polymer solution is pumped into a high-pressure vessel with supercritical CO₂, according to the instructions of Kang et al. (ibid.), by means of a HPLC pump, causing the nanoparticles to precipitate out immediately.

a. Coating of a Medical Product According to the Invention by Means of a Spraying Method

In the coating of a medical product to be used according to the invention, preferably a commercially available stent or angioplasty balloon, the active substance particles to be used according to the invention, preferably the nanoparticles produced under A), can be applied to the surface of the medical product, together with a binder in a suitable solvent, where the solvent can (partially) dissolve the binder, but not the active substance particles, preferably nanoparticles. Usually, all water-soluble polymers and oligomers, such as carbohydrates, for example, preferably sugar and polysaccharides, preferably hyaluronic acid, and/or gelatins, are suitable as solvents.

Furthermore, attention must be paid to ensure that the active substance particles to be used according to the invention, preferably nanoparticles, do not plug up the nozzle of the coating apparatus, i.e. that the maximal particle diameter of the active substance particles to be used according to the invention is preferably smaller than the gap width of the nozzle of the coating apparatus.

### Production Example B1

A suspension of the active-substance-charged nanoparticles (<200 nm) produced according to production method A), in ultra-pure water (10 wt.-%), is batched up at RT. If necessary, a wetting agent or dispersant (0.5-3 wt.-%), such as lecithin, AOT (his-(2-ethyl-1-hexyl) sulfosuccinate), polyethylene oxide/polypropylene oxide block copolymer, tetra-alkyl ammonium salts, or silicates can be added.

A stent made available is cleaned of dust and residues and clamped into a suitable stent coating apparatus (DES Coater, developed by Biotronik). Using an airbrush system (from EFD or Spraying System), the rotating stent is coated with the nanoparticle suspension on half its side, under constant ambient conditions (room temperature; 42% relative humidity). At a nozzle distance of 20 mm, a stent having a length of 18 mm is usually coated after about 10 min. After the intended layer mass has been reached, the stent is dried in a vacuum for 5 min, before the uncoated side is coated in the same manner, after the stent has been turned and clamped in again. The completely coated stent is dried for 36 h at 40° C., in a vacuum oven (Vakucell; from MMM).

### Production Example B2

A stent made available is cleaned of dust and residues and clamped into a suitable stent coating apparatus (DES Coater, developed by Biotronik). Using an airbrush systems (from EFD or Spraying System), the rotating stent is coated with organic adhesive material on half its side, under constant ambient conditions (room temperature; 42% relative humidity). The active substance nanoparticles are subsequently sprayed onto the mural side of the stent by means of a solvent-free method, in an airbrush system, as a dry powder. Depending on the type of adhesive, drying takes place in a vacuum or by means of cross-linking using UV light.

### Production Example B3

Coating of a commercially available angioplasty balloon that is made available takes place in accordance with one of the Production Examples B1 or B2.

b. Coating of a Medical Product According to the Invention by Means of a Dipping Method:

### Production Example C1

A stent that is made available is dipped into a stabilized suspension including active substance particles, preferably the nanoparticles produced according to Production Method A), hydrophilic polymer (carbohydrates, hyaluronic acid, and/or gelatin), wetting agent or dispersant additive, as well as ultra-pure water, and subsequently dried in a vacuum.

### Production Example C2

Coating of a commercially available angioplasty balloon that is made available takes place in accordance with Production Example C1.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

What is claimed is:

1. A medical product that can be implanted or introduced into a vascular system of a human or animal organism, having an active substance coating on the surface of said medical product, wherein
a) said active substance coating comprises one or more separate active substance particles, comprising one or more active substances and one or more polymers, having a particle diameter less than or equal to 30 μm,
b) said active substance particle(s) can be detached from said coating of said medical product under physiological conditions in the vascular system, and
c) said detached active substance particle(s) essentially does/do not release said active substance(s) at a pH in the range of 7.3-7.5.

2. The medical product according to claim 1, wherein said polymer(s) of said active substance particle is/are selected from among pH-sensitive degradable polymers.

3. The medical product according to claim 2, wherein said pH-sensitive degradable polymer is selected from among the group consisting of poly-β-amino esters), poly-(ethylene oxide)-modified poly-β-amino esters), polylactides, poly-(lactide-co-glycolides), modified cyclodextrins, modified carbohydrates and/or other polyesters.

4. The medical product according to claim 1, wherein said active substance particles on the surface of said medical product are (i) incorporated into a degradable polymer layer or (ii) are present on the surface of said medical product as an active substance coating by means of adhesive material.

5. The medical product according to claim 4, wherein (i) said degradable matrix is selected from among one or more materials of the group consisting of polydioxanones; polycaprolactones, polyglycolides; polylactides as well as blends, copolymers and tripolymers of them; polysaccharides; polyhydroxyvalerates; ethyl vinyl acetates; polyethylene oxides; polyphosphoryl cholines; fibrin; albumin; and/or polyhydroxybutyric acids as well as their blends.

6. The medical product according to claim 4, wherein (ii) said adhesive material is selected from among the group consisting of carbohydrates; (soy) lecithin; (oligo)peptides and quickly degradable polyesters.

7. The medical product according to claim 1, wherein said active substances are selected from among the group consisting of antiphlogistics; cytostatics; immune suppressives; thrombocyte aggregation inhibitors; estrogens; lipid regulators; vasodilators; calcium channel blockers; calcineurin inhibitors; anti-inflammatories; anti-allergies; oligonucleotides; endothelium-forming agents; steroids; proteins/peptides; proliferation inhibitors; analgesics and anti-rheumatics; endothelin receptor antagonists; rho-kinase inhibitors; RGD-peptides; and organic gold compounds.

8. The medical product according to claim 1, wherein said medical product is an active-substance-eluting stent.

9. The medical product according to claim 8, wherein said active-substance-eluting stent is a degradable metal stent.

10. The medical product according to claim 8, wherein said active substance coating is applied to the mural surface of the stent.

11. The medical product according to claim 1, wherein said medical product is an active-substance-eluting angioplasty balloon.

12. A method for the production of a medical product that can be implanted or introduced into a vascular system of a human or animal organism, having an active substance coating, wherein
a) one or more separate active substance particles is/are made available, which comprise one or more active substances and one or more polymers, and have a particle diameter less than or equal to 30 μm,
b) a base body of a medical product is made available, and
c) said base body from step b) is coated with said active substance particles from step a), in such a manner that said active substance particles can be detached under physiological conditions in the vascular system, in the coated state, but said active substance(s) is/are essentially not released from said detached active substance particles at a pH in the range of 7.3 to 7.5.

13. An active substance coating for a medical product that can be implanted or introduced into a vascular system of a human or animal organism, wherein
a) said active substance coating comprises one or more separate active substance particles comprising one or more active substances and one or more polymers, having a particle diameter less than or equal to 30 μm,
b) said active substance particle(s) can be detached under physiological conditions in the vascular system, from said coating of said medical product, and
c) said detached active substance particle(s) essentially does/do not release said active substance(s) in a pH range of 7.3-7.5.

14. A method for treatment or a stenosis, a calcified or soft plaque, or an aneurysm in the vascular system of a human or animal organism, wherein
a) the active-substance-coated medical product according to claim 1 is made available, and
b) implanted or introduced into the vascular system of the human or animal organism.
